(19) United States
(12) Patent Application Publication
(10) Pub. No.: US 2017/0319122 A1
(43) Pub. Date: Nov. 9, 2017

(54) A SYSTEM AND A METHOD FOR GNERATING STRESS LEVEL AND STRESS RESILIENCE LEVEL INFORMATION FOR AN INDIVIDUAL

(71) Applicant: GLOBAL STRESS INDEX PTY LTD, Sydney, New South Wales (AU)

(72) Inventors: Travis Leigh Wild, Sydney (AU); Stephen Aaron Foster, Sydney (AU)

(21) Appl. No.: 15/525,887

(22) PCT Filed: Nov. 11, 2015
(86) PCT No.: PCT/AU2015/050703

§ 371 Date: May 10, 2017

(30) Foreign Application Priority Data
Nov. 11, 2014 (AU) 2014904521

(51) Int. Cl. A61B 5/16; A61B 3/113; A61B 5/00; A61B 5/20; G09B 7/10; A61B 5/0205; A61B 5/0476; A61B 5/0488; G09B 19/00; A61B 5/08; A61B 5/053; A61B 5/024; A61B 5/021; A61B 5/11; A61B 5/145

(52) U.S. Cl. CPC A61B 5/165; G09B 19/00; A61B 3/113; A61B 5/743; A61B 5/7246; A61B 5/4884; A61B 5/4806; A61B 5/4803; A61B 5/448; A61B 5/441; A61B 5/4277; A61B 5/20; A61B 5/168; G09B 7/10; A61B 5/02055; A61B 5/0476; A61B 5/0488; A61B 5/0816; A61B 5/0531; A61B 5/02405; A61B 5/021; A61B 5/1112; A61B 5/112; A61B 5/14542; A61B 5/14546

(57) ABSTRACT

A system and a method for generating stress level information for an individual and stress level resilience information for an individual, which includes a stress information processing module configured to process stress information for the individual, the stress information for the individual comprising at least two of psychometric information for the individual, physiological information for the individual, behavioural information for the individual, and cognitive function information for the individual.

### CROSS REFERENCE TO RELATED APPLICATIONS

This application claims the benefit of international application PCT/AU2015/050703 with an international filing date of Nov. 11, 2015 which claims priority from application 2014904521 filed in Australia on Nov. 11, 2014, the disclosures of both of which are specifically incorporated herein by reference.

### TECHNICAL FIELD

The disclosure herein generally relates to a system and a method for generating stress level information for an individual, as well as a system and method for generating stress resilience level information.

### BACKGROUND

Stress in humans can be categorised as either acute (short-term) or chronic (long-term).

Examples of sources of acute stress include physical activities to which the individual is not accustomed, an upset in a relationship, a bereavement, public speaking, or having a higher than usual workload for days, weeks or months. People normally adapt to acute stress and then recover from it as soon as the stress passes. Because of this ability to adapt and recover, acute stress per se may not be as damaging to our wellbeing as chronic stress.

However, stress resilience can be an indication of underlying damage occurring to a person's wellbeing. Stress resilience is a person's ability to respond to an acute stress event or an acute stress state. For example, one particularly important aspect of stress level resilience is the time taken for the individual acute stress elements and indicators, either singular or in combination, to return to ‘unstressed’ or baseline levels following any particular stressful event.

As an example, if a person becomes acutely stressed—exercising or giving a presentation at work—their stress indicators such as heart rate, heart rate variability, sweat (skin conductivity) and so on, would elevate. These stress measures can be detected and recorded.

When the stress subsides, these indicators should return to their previous baseline over the next 15 to 30 minutes. However, in a person with ‘diminishing stress resilience levels’, their stress response can be more accelerated (more ‘excitable’), can be heightened or accentuated (more ‘reactive’), and take longer to return to ‘normal’ with their stress ‘half-life’ or ‘resolution to baseline’ taking longer (slower resolution). The more rapid and accentuated the response and the longer the recovery time, the less stress resilience the individual has, even if their stress measures do eventually return to ‘normal’ or ‘baseline’ levels.

An individual who shows lower stress resilience levels is more prone to ‘cracking’, ‘breaking down’ or developing stress-related conditions (such as PTSD) under additional pressure.

Chronic stress is stress that is ongoing over many months or years, such as being in prison, having a very high workload over a long period of time, or the cumulative build-up of acute stress over many years that does not fully resolve in the body and brain after the stressful situations dissipate. Chronic stress is known to contribute to many aspects of poor health.

Further, when an individual is becoming overwhelmed or ‘burnt out’ by a singular stress or chronic stress, their acute stress response can become diminished. They can have a delayed or reduced response of some acute stress response components to an acute stress such as exercise (sluggish or minimal response) that should ordinarily elicit a stress response.

### Range and Sensitivity

Individual stress tests may display a low range i.e. they only detect a relatively low percentage of the total number of stressed individuals, examples of which include questionnaires about an individual's subjective experience (referred to as psychometric questionnaires) developed predominantly by psychologists and biological or physiological tests (such as heart rate, blood pressure, cortisol levels in saliva), developed by medical researchers or physiologists.

Stress measurement devices may be insensitive to individuals who are more chronically stressed, particularly in the early stages of developing chronic stress. A measurement of blood pressure may detect the initial acute stress, the blood pressure reading may soon return to normal. Blood pressure measurements may fail to detect the early stages of chronic stress build-up. It is not until a person's homeostatic mechanisms are being overwhelmed and cannot adapt any further that their blood pressure becomes permanently elevated. By this time, the individual may be very stressed and significant damage may have occurred.

Physiological measures such as blood pressure may only discover a small number of the chronically stressed (they have low range) and then only those that are very stressed (they have low sensitivity).

Other physiological methods of measuring stress generally may exhibit large variations between individual persons. For example, one unstressed person's salivary cortisol levels can be over three times higher than another unstressed person. In order for a person to be considered stressed using physiological methods, the person needs to measure well outside the normal range. This means that a person needs to be very stressed in order to be detected using this type of screening.

Psychometric questionnaires generally may be insensitive to early stages of stress. A person using these tests needs to be very stressed in order to be detected in initial screening. For example, one of the industry standard questionnaires, The Depression, Anxiety and Stress Scale, measures the state of irritability as its stress indicator. A person needs to be very chronically stressed to become irritable (low sensitivity), and even then only a small percentage of the very chronically stressed may experience themselves as irritable (narrow range).

The same may be true for the cognitive function tests indicative of stress: they may be insensitive to people in the early stages of stress, and even in advanced cases may only detect a small portion of the extremely stressed. Individual people generally have different capacities in regards to memory, reaction times, decision-making and so on. A general screening test for stress based upon cognitive function alone may not useful as it may only detect the extremely dysfunctional or extremely stressed. At best cognitive function tests for stress by themselves may be only of use in a baseline test-retest format.

Because of this low sensitivity to chronically stressed individuals, even in the event that the test being applied may be in the range that can detect the type of stress indicator that the person is going to show, the existing methods for measuring stress are generally unable to detect individuals in the early stages of becoming stressed. By the time the stress is detected the opportunity for early intervention (which would result in better outcomes at less expense) may be lost.

### Requirement for Baselines in the Prior Art

Baselines may be used in an attempt to compensate for variances between people in physiological stress tests and for the lack of sensitivity for detecting chronic stress in both the physiological tests and existing psychometric questionnaires. An individual may be measured at one point in time and then, once the first baseline test is established, future measurements may be compared to the first test.

For example, as discussed above, one unstressed individual can have salivary cortisol levels at more than three times another unstressed person. If you take a baseline measure from one person and their salivary cortisol level falls within normal range, it tells you nothing about that person's chronic stress levels. They may be utterly unstressed, with no chronic stress build up, or—with exactly the same score—they may have been accommodating chronic stress for decades, adapting but using up their reserve capacities. This first test may only able to detect chronic stress, as stated above, in the very chronically stressed.

Once a baseline is established for a person, second or subsequent tests are able to reveal fluctuations from the initial baseline point. These can be useful for determining whether a person is becoming either more or less chronically stressed, but they still do not accurately determine whether their first test was truly ‘unstressed’ or not.

For example, when a person is more acutely stressed they may type on their keyboard or smartphone more quickly or more forcefully. Over time if a stress or resilience stress measure detects faster or more forceful typing style when that person's other stress indicators are elevated, then the detection of this type of typing style change in future may be useful as a very sensitive indicator of stress accumulation. Further, if a stress or resilience stress behaviour such as faster or more forceful typing style is determined in a person and then that person experiences a significant stress or trauma, if the faster or more forceful typing behaviour remains constant for weeks or months it may be a very accurate and useful determination of a retained stress response or the development of Post Traumatic Stress Disorder in the individual. Because many of these behaviours are not intentional conscious activities they may offer very significant gains in being able to determine stress or resilience stress changes even when the individual is unaware of being stressed or reticent to report being stressed.

### SUMMARY

Disclosed herein is a method for generating stress level information for an individual. The method comprising the step of, in a processor, processing stress information for the individual comprising at least two of psychometric information for the individual, physiological information for in the individual, behavioural information for the individual, and cognitive function information for the individual.

An embodiment comprises the step of receiving the stress information.

In an embodiment, the step of processing the stress information comprises the step of correlating at least one stress indicator in one of the psychometric information, the physiological information, the behavioural information, and the cognitive function information with at least one other stress indicator in at least one other of the psychometric information, the physiological information, the behavioural information, and the cognitive function information.

In an embodiment, the step of processing the stress information comprises the step of determining a stress feature that recurs within the stress information. Further stress information may be received for the individual, and the presence of the stress feature within the further stress information confirmed. An alert may be generated indicative of the presence of the stress feature when the presence of the stress feature within the further stress information is so confirmed.

In an embodiment, the stress information comprises the psychometric information. The psychometric information may be generated by the individual responding to an electronic stress questionnaire. The questionnaire is preferably in two parts, each comprising a different set of predefined questions, whereby the individual is presented with the second set of questions based on predetermined criteria correlating with the answers provided to the first set of questions. The psychometric information may be indicative of a plurality of chronic stress indicators.

In an embodiment, the stress information comprises the physiological information.

An embodiment comprises the step of generating the physiological information. The step of generating the physiological information may comprise the step of generating information for each of a plurality of physiological functions in the individual. The step of generating information indicative of stress in each of a plurality of physiological functions in the individual may comprise generating at least one of heart rate information, heart rate variability information, respiratory rate information, respiratory rate variability information, blood pressure information, physical movement information, cortisol level information, a skin conductivity information, skin temperature information, blood oxygen saturation information, surface electromyography information, electroencephalography information, blood information, saliva information, and urine information.

In an embodiment, the stress information comprises the behavioural information.

An embodiment comprises the step of generating the behavioural information. The step of generating the behavioural information may comprise at least one of the steps of: generating eye movement information indicative of eye movement of the individual; generating location information indicative of a plurality of locations the individual has been; generating nearby device information indicative of the nearby presence a plurality of devices of a plurality of people to the individual; generating internet browsing history information for the individual; generating keystroke rate, cadence, typing style, pressure or ‘force’ detection information for the individual; generating voice analysis, including tone, cadence, word and phrase detection information for the individual; generating telephone usage analysis, including call time, numbers dialed and time of day calls placed information for the individual; generating driving style, including steering inputs, acceleration, deceleration, braking, speed of driving, brake and accelerator force and data from door pressure sensor information for the individual; generating movement, body temperature, television usage, including channels watched, time watched and eye movement whilst watching, refrigerator analytics, heating and cooling analytics information for the individual; generating bicycle data, including pedal force, pedaling cadence, acceleration, speed, routes taken, GPS data, altimeter data, time on bicycle, pedometer data information for the individual; generating pedometer data and gait analysis information for the individual; generating application usage information indicative of application usage by the individual; generating media consumption information indicative of media consumption by the individual; generating spending behaviour information indicative of the individual's spending behaviour; generating food choice information indicative of a plurality of food choices made by the individual; generating social outing information indicative of the individual's social outing activity; generating productivity information indicative of the individual's ability to work and be productive; and generating leave information indicative of leave taken by the individual.

The stress information may comprise the cognitive function information.

An embodiment comprises the step of generating the cognitive function information. The step of generating the cognitive information may comprise at least one of the steps of: generating memory function information indicative of a memory function of the individual; generating reaction time information indicative of a reaction time of the individual; generating attention ability, peripheral vision and comprehension ability of the individual; and generating decision-making ability information indicative of a decision-making ability of the individual.

A method defined by any one of the preceding claims wherein the step of processing the stress information comprises the steps of generating: a physiological and/or physical stress score for the individual using the stress information; a mental stress score for the individual using the stress information; an emotional stress score for the individual using the stress information; a life stress score for the individual using the stress information.

An embodiment comprises the step of displaying on an electronic display graphical representations of: the physiological and/or physical stress score for the individual; the mental stress score for the individual; the emotional stress score for the individual; and the life stress score for the individual. The step of displaying on an electronic display comprises the step of displaying a token for each of: the physiological and/or physical stress score; the mental stress score; the emotional stress score; and the life stress score; wherein the size of each token is indicative of a respective stress score magnitude.

In an embodiment, the token for each of the physiological and/or physical stress score, the mental stress score, the emotional stress score; and the life stress score are simultaneously displayed

An embodiment comprises the step of generating an acute stress score indicative of a magnitude of acute stress for the individual.

An embodiment comprises the step of generating a stress resilience score indicative of a response to acute stress for the individual. Preferably, the stress resilience score is indicative of one or more of the time taken for the individual to respond to an acute stress event, if the individual exhibits any response to an acute stress event, and if so, the level of response exhibited by the individual to an acute stress event and the time taken for the individual's stress information to return to baseline levels following a period of acute stress

An embodiment comprises the step of generating a chronic stress score indicative of a magnitude of chronic stress for the individual.

An embodiment comprises the step of analysing the stress level information, which can indicate if the individual is suffering from a psychological condition, including post-traumatic stress disorder, depression, anxiety, suicide/self-harm risk or prediction, bipolar disorder, attention deficit hyperactivity disorder, sleep disorders and addictive traits.

Disclosed herein is a system for generating stress level information for an individual. The system comprises a stress information processing module configured to process stress information for the individual, the stress information for the individual comprising at least two of psychometric information for the individual, physiological information for the individual, behavioural information for the individual, and cognitive function information for the individual.

An embodiment comprises a stress information receiver configured to receive the stress information.

In an embodiment, the stress information processing module is configured to correlate at least one stress indicator in one of the psychometric information, the physiological information, the behavioural information, and the cognitive function information with at least one other stress indicator in at least one other of the psychometric information, the physiological information, the behavioural information, and the cognitive function information.

In an embodiment, the stress information processing module is configured to determine a stress feature that recurs within the stress information. The stress information processing module may be configured to confirm the presence of the stress feature within further stress information for the individual.

An embodiment comprises an alert generation module configured to generate an alert indicative of the presence of the stress feature when the presence of the stress feature within the further stress information is so confirmed.

In an embodiment, the stress information comprises the psychometric information.

An embodiment comprises a psychometric information generator configured to generate psychometric information for the individual.

In an embodiment, the psychometric information generator is configured to execute an electronic psychometric questionnaire for the individual.

In an embodiment, the psychometric information is indicative of a plurality of chronic stress indicators.

In an embodiment, the stress information comprises the physiological information.

An embodiment comprises a physiological information generation system configured to generate the physiological information. The physiological information generation system may be configured to generate information for each of a plurality of separate physiological functions in the individual. The physiological information generation system may be configured to generate information for each of a plurality of separate physiological functions in the individual is configured to generate at least one of a heart rate information, heart rate variability information, respiratory rate information, respiratory rate variability information, blood pressure information, physical movement information, cortisol level information, skin conductivity information, skin temperature information, blood oxygen saturation information, surface electromyography information, electroencephalography information, blood measurement information, saliva measurement information, and urine measurement information.

In an embodiment, the stress information comprises the behavioural information.

An embodiment comprises a behavioural information generator configured to generate the behavioural information.

In an embodiment, the behavioural information generator comprises at least one of: an eye movement information generator configured to generate eye movement information indicative of eye movement of the individual; a location information generator configured to generate location information indicative of a plurality of locations the individual has been; a nearby device information generator configured to generate nearby device information indicative of the nearby presence a plurality of devices of a plurality of people to the individual an internet browsing history generator configured to generate internet browsing history for the individual; a keystroke generator configured to generate rate, cadence, typing style, pressure or ‘force’ detection information for the individual; a voice analysis generator configured to generate tone, cadence, word and phrase detection information for the individual; a telephone usage analysis generator configured to generate call time, numbers dialed and time of day calls placed information for the individual; a driving style generator configured to generate steering inputs, acceleration, deceleration, braking, speed of driving, brake and accelerator force and data from door pressure sensor information for the individual; a movement generator configured to generate body temperature, television usage, including channels watched, time watched and eye movement whilst watching, refrigerator analytics, heating and cooling analytics information for the individual; a bicycle usage data generator configured to generate pedal force, pedaling cadence, acceleration, speed, routes taken, GPS data, altimeter data, time on bicycle, pedometer data information for the individual; a pedometer and gait analysis generator configured to generate pedometer data information for the individual; an application usage information generator configured to generate information indicative of application usage by the individual; a media consumption information generator configured to generate media consumption information indicative of media consumption by the individual; a spending behaviour information generator configured to generate spending behaviour information indicative of the individual's spending behaviour; a food choice information generator configured to generate food choice information indicative of a plurality of food choices made by the individual; a social outing information generator configured to generate social outing information indicative of the individual's social outing activity; generating productivity information indicative of the individual's ability to work and be productive; and a leave information generator configured to generate leave information indicative of leave taken by the individual.

In an embodiment, the stress information comprises the cognitive function information.

An embodiment comprises a cognitive function generator configured for generating the cognitive function information. The cognitive function generator may comprise at least one of: a memory function information generator configured to generate memory function information indicative of a memory function of the individual; an attention ability, peripheral vision capability and comprehension ability generator to generate information indicative of the ability of the individual; a reaction time information generator configured to generate reaction time information indicative of a reaction time of the individual; and a decision-making ability information generator configured to generate decision-making ability information indicative of a decision-making ability of the individual.

In an embodiment, the stress information processing module is configured to generate at least one of: a physiological and/or physical stress score for the individual using the stress information; a mental stress score for the individual using the stress information; an emotional stress score for the individual using the stress information; a life stress score for the individual using the stress information.

An embodiment comprises a display and the configured to display on the electronic display graphical representations of: the physiological and/or physical stress score for the individual; the mental stress score for the individual; the emotional stress score for the individual; and the life stress score for the individual.

An embodiment is configured to display on the electronic display a token for each of: the physiological and/or physical stress score; the mental stress score; the emotional stress score; and the life stress score; wherein the size of each token is indicative of a respective stress score magnitude.

An embodiment is configured to simultaneously display the token for each of the physiological and/or physical stress score, the mental stress score, the emotional stress score, and the life stress score.

In an embodiment, the stress information processing module is configured to generate an acute stress score indicative of a magnitude of acute stress for the individual.

In an embodiment, the stress information includes a stress resilience score indicative of a response to acute stress for the individual. Preferably, the stress resilience score is indicative of one or more of the time taken for the individual to respond to an acute stress event, if the individual exhibits any response to an acute stress event, and if so, the level of response exhibited by the individual to an acute stress event and the time taken for the individual's stress information to return to baseline levels following a period of acute stress.

In an embodiment, the stress information processing module is configured to generate a chronic stress score indicative of a magnitude of chronic stress for the individual.

In an embodiment, the stress level information can indicate the individual is suffering from a psychological condition, including post-traumatic stress disorder, depression, anxiety, suicide/self-harm risk or prediction, bipolar disorder, attention deficit hyperactivity disorder, sleep disorders and addictive traits.

Disclosed herein is non-transitory processor readable tangible media including program instructions which when executed by a processor causes the processor to perform a method disclosed above.

Disclosed herein is a computer program for instructing a processor, which when executed by the processor causes the processor to perform a method disclosed above.

Any of the various features of each of the above disclosures, and of the various features of the embodiments described below, can be combined as suitable and desired.

### BRIEF DESCRIPTION OF THE FIGURES

Embodiments will now be described by way of example only with reference to the accompanying figures in which:

FIG. 1 shows a block diagram of an embodiment of a system for generating stress level information for an individual in the form of a human.

FIG. 2 is a block diagram of another representation of the system of FIG. 1.

FIG. 3 is a graphical representation of example results generated by the system of FIG. 1.

FIG. 4 shows a screen shot from a psychometric information collection tool.

### DESCRIPTION OF EMBODIMENTS

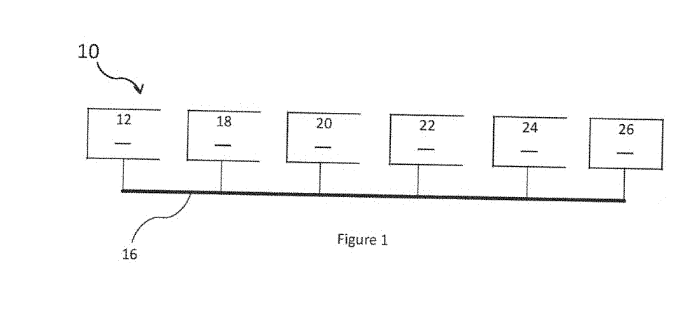

FIG. 1 shows a block diagram of an embodiment of a system for generating stress level information for an individual in the form of a human, the system being generally indicated by the numeral 10.

The system 10 is configured to execute the steps of an embodiment of a method described herein. The method may be coded in a program for instructing the processor 10. The program is, in this embodiment stored in the non-volatile memory 20, but could be stored in FLASH, EPROM or any other form of tangible media within or external of the system 10. The program generally, but not necessarily, comprises a plurality of software modules that cooperate when installed on the system so that the steps of an embodiment of the method are performed. The software modules, at least in part, correspond to the steps of the method or components of the system 10 described herein. The functions or components may be compartmentalised into modules or may be fragmented across several software and/or hardware modules. The software modules may be formed using any suitable language, examples of which include C++ and assembly. The program may take the form of an application program interface or any other suitable software structure.

The system 10 includes a suitable microprocessor 12 such as, or similar to, the INTEL XEON or AMD OPTERON micro processor connected over a bus 16 to memory which includes a suitable form of random access memory 18 of around 1 GB, or generally any suitable alternative capacity, and a non-volatile memory 20 such as a hard disk drive or solid state non-volatile memory (e.g. NAND-based FLASH memory) having a capacity of around 500 Gb, or any alternative suitable capacity. Alternative logic devices may be used in place of the microprocessor 12. Examples of suitable alternative logic devices include application-specific integrated circuits, field programmable gate arrays (FPGAs), and digital signal processing units. Some of these embodiments may be entirely hardware based. The system 10 has at least one communications interface. In this embodiment, the at least one communications interface 22 comprises a network interface in the form of an Ethernet card, however generally any suitable network interface may be used, for example a Wi-Fi module. The network interface 22 is configured, in this but not necessarily all embodiments, to send and receive information in the form of data packets. The data packets are in the form of Ethernet frames that have an Internet Protocol (IP) packet payload. The IP packets generally have a Transmission Control Protocol (TCP) segment payload, although any suitable protocol may be used. In the present embodiment, the TCP segments may carry hypertext transfer protocol (HTTP) data, for example web page information in HTTP, for example, or a HTTP request or a HTTP response. The HTTP data may be sent to a remote machine. In alternative embodiments, however, proprietary protocols and applications may be used, or generally any suitable protocol (for example SONET, Fibre Channel) or application as appropriate.

The system 10 has a stress information processing module configured to process stress information for the individual, the stress information for the individual comprising at least two of psychometric information for the individual, physiological information for the individual, behavioural information for the individual, and cognitive function information for the individual.

The system 10, in this but not all embodiments, generates a stress profile indicative of the magnitude and form of stress experienced by the user at the time of testing. The system or stress profiler 10 processes at least two of the following types of stress information:
psychometric information indicative of stress in the user,
physiological information indicative of stress in the user,
behavioural information indicative of stress in the user, and
cognitive function information indicative of stress in the user.

The combination of multiple types of stress information helps to increase the sensitivity and characterise the form of stress being experienced by a user. Being able to characterise the form of stress enables more targeted and effective treatments to be developed and prescribed

At a minimum the stress profiler 10 processes two of the types of stress information. In one embodiment, the stress profiler 10 processes psychometric and physiological information. However, the accuracy and sensitivity of the stress profiler 10 generally increases when more of the types of stress information are processed. The stress profiler 10 may therefore process three of the four, or even all four of the four types of stress information.

The reason is that some forms of stress information tend to be more sensitive to acute stress and some tend to be more sensitive to chronic stress. For example, if only physiological information are measured, then chronic stress may not be identified at all.

In one embodiment, the stress profiler 10 processes psychometric, physiological and behavioural information. In another embodiment, the stress profiler 10 processes all four of the types of stress information (psychometric, physiological and behavioural, and cognitive function).

The stress profiler 10 can include a learning function, which recognizes patterns of stress information associated with previous periods of stress. Over time, the learning function progressively improves the accuracy and speed of stress profiling for a user.

The stress profiler 10 can also include a predictive function which identifies patterns of stress information indicative of the early signs of stress and notify the user early. For example, the stress profiler 10 may correlate a pattern of eye movement with physiological or psychometric indicators of stress in the particular user, and notify the user when those eye movements are detected—before serious symptoms arise.

Further, the predictive function can identify patterns of stress information which are indicative of the potential for stress to arise in the future, and notify the user accordingly. For example, the behavioural information may detect that the user has travelled to a location which has been previously associated with a stressed state, and therefore warn the user about it.

### The Psychometric Information

The psychometric information comprises responses to a questionnaire about the user's subjective experience of stress.

Preferably, the questionnaire asks questions about a wide range of signs or symptoms associated with the human stress response, particularly those aspects that are connected to the accumulation of chronic stress.

The greater the number and severity of chronic stress indicators, the higher the probability that they are linked to a singular underlying cause (chronic stress) rather than just occurring in the same person coincidentally. For example, one person might experience occasional tight shoulders, digestive issues and a rash that comes and goes. These symptoms, individually or even all three together, could be occurring for a number of different reasons and have nothing to do with a person developing chronic stress. However, if they also had persistent headaches, difficulty getting to sleep at night and frequent viral infections, it is beginning to tell a different story: they now have six indicators of chronic stress.

A wide range of questions is desirable because it helps to detect stress in more people. Stress manifests differently in different people, depending on many factors such as genetic makeup, fitness, constitution, health history, so a wide range of questions is more likely to detect more manifestations of stress.

The answers to some questions may correlate strongly with other questions, forming statistically coherent factors (determined through a psychometric statistical method called Exploratory Factor Analysis). Each statistically coherent factor may be indicative of a particular type of stress being experienced by an individual.

To best obtain a psychometric stress measure a long-form′ and ‘short form’ questionnaire has been developed as part of this invention. In use, the psychometric stress measure will be deployed in a two stage approach, which incorporate both the ‘long form’ and the ‘short form’ questionnaires. During the first stage, an initial set of questions are posed to the individual. In a preferred embodiment, the questions that form part of this first stage will take approximately three minutes for the individual to complete. If the individual scores above a certain cut-off level, or in pre-set patterns, then the individual will be prompted to complete another block of questions, which constitutes the second stage of the questionnaire. In a preferred embodiment, this second set of questions will take approximately four to five minutes to complete. It is also envisaged that the individual will have the option (if desired) to complete the second stage set of questions, no matter their score when completing the first stage of questions.

In one embodiment, the psychometric information comprises responses to a questionnaire which asks individuals about their subjective experience of stress-related signs, symptoms or indicators across four forms of stress:
physical/physiological stress,
mental stress,
emotional stress, and
current perceived life stress.

The questionnaire uses multiple lines of questioning to cover the range of known subjective states associated with stress—particularly those noted to be indicative of chronic stress in humans. The questionnaire indicates which form of stress an individual scores more highly in. The person can then be given feedback about which type of intervention(s) are most likely to produce the greatest benefit for the person and track the results over time.

By combining the psychometric information with other types of stress information, such as physiological, behavioural or cognitive function information, the sensitivity and range to of the stress profiler 10 is increased. Also, the other types of stress information help to detect those people who do not respond well to questionnaires.

### The Physiological Information

There are many known physiological indicators of stress in humans. Many lie detectors are based on measuring multiple physiological indicators of stress.

Where physiological information is used by the stress profiler 10, the accuracy and sensitivity of the stress profiler 10 generally increases when the physiological information includes measurements of more than one physiological parameter.

Examples of different measurements which may be used to provide physiological information include heart rate measurements, heart rate variability measurements, respiratory rate measurements, respiratory rate variability measurements, blood pressure measurements, physical movement observations, cortisol level measurements (measured in blood or saliva), skin conductivity measurements, skin temperature measurements, skin or hair analysis, DNA analysis, blood oxygen saturation measurements, surface electromyography (surface EMG) measurements, electroencephalography (EEG) measurements and measurements other physiological indicators of stress able to be determined by analysis of a person's blood, saliva or urine. The saliva, blood, urine, skin, hair and DNA measurements can be carried out through conventional laboratory testing or via nanotechnology, where for example, nanotechnology sensors can be used for single-blood drop measures, can be incorporated in a transdermal patch, can be injected subcutaneously or circulate within the body of the individual or may incorporate the use of a subcutaneously embedded microchip or wire-enabled sensor.

Furthermore, ‘smart clothing’ can also be utilised, which can include pants/trousers, underwear, socks, shoes, shirts/T-shirts, gloves, hats/caps/helmets, glasses, watches, smart-watches, wrist and ankle bands, as well as adhesive patches. The ‘smart clothing’ is embedded with various sensors, including electrical signal, conductivity (galvanic conductance and resistance), accelerometers, force, temperature, chemical sensors and nanotechnology sensors can be used to provide physiological information.

The physiological measurements may be selected in accordance with their sensitivity and relevance as well as their ease of application as a screening device.

### The Behavioural Information

Where behavioural information is used by the stress profiler 10, the accuracy and sensitivity of the stress profiler 10 generally increases when the behavioural information includes measurements of more than one behavioural parameter. These behaviours may be generally known to be indicative of stress in humans, or they may be individual traits of the user. For example, a user may exhibit a particular pattern of eye movement, pace up and down, or visit a particular location when stressed.

The stress profiler 10 may progressively acquire behavioural information by progressively correlating behaviours with other forms of stress information, such as cognitive function information, psychometric information or physiological information.

Examples of different measurements or behavioural observations which may be used to provide behavioural information include eye movement patterns, social interactions, the types of websites visited, the types of apps used, the news topics read, spending behaviour, food choices, social outings, taking holidays, and so on.

Data can be obtained from smartphones, smart-watches or other wearable devices, tablets and computers, which can be measured by the accelerometer, gyroscope, altimeter, GPS, NFC (proximity to other devices, enhanced location specificity), Bluetooth (proximity to other devices, enhanced location specificity), Wi-Fi (proximity to other devices, enhanced location specificity). Other inputs can be measured such as, keystroke rate, cadence, typing style, pressure or ‘force’ detection (keypad, trackpad, screen pressure sensor), voice analysis (tone, cadence, word and phrase detection), phone usage, including call time, numbers dialed, time of day calls placed, Application (‘app’) usage, including specific applications used, duration of usage, time of day apps used, in-app analytics (use characteristics within any app), keyword searches, word and phrase usage (usually applied within word processing, email, messaging and social media applications but not limited to these), eye movement patterns, gait and posture analysis and purchasing history.

Other behavioural observations can be obtained from car/driving/riding style, which include steering inputs, acceleration, deceleration, braking, speed of driving, brake and accelerator force, door pressure sensors and other vehicle sensors.

Further behavioural observations can be obtained from home or office sensors, which can measure movement, body temperature, television usage (channels watched, time watching, eye movement), refrigerator analytics, heating and cooling analytics and other ‘smart home’ analytics.

Additionally, behavioural observations can also be obtained from other measurement devices such as bicycle meters (pedal force, pedaling cadence, acceleration, speed, routes taken, GPS, altimeter, time on bicycle, and so on), pedometers, gait analysis measures and other measurements obtained from ‘smart clothing’, which includes pants/trousers, underwear, socks, shoes, shirts/T-shirts, gloves, hats/caps/helmets, glasses, watches, smart-watches, wrist and ankle bands, as well as adhesive patches.

### Behavioural Analysis Based on Physiological Measures

The various measurements, which can be used to determine the physiological information, and specifically those that are described above under the ‘Physiological Information’ section can be used in a ‘behavioural analytic’ pattern beyond a specific ‘stress and resilience’ physiological manner.

As an example of this; a heart rate meter is ordinarily used to detect heart rate, heart rate variability, return to baseline after exercise or stress event and so on. Measuring these aspects might return an ‘unstressed’ physiological measure in purely physiological terms, but it may be that as a person is becoming increasingly stressed the individual spends a greater or lesser amount of time exercising, or they exercise more or less frequently. These ‘behavioural physiological indicators’ could well be the most reliable early indicator for chronic stress build up.

Another example is an individual's physiological sleep measurements. For example, a sleep sensor might detect ‘normal’ sleep patterns (depth, timing of sleep cycles, and so on) but a behavioural analysis of sleep might correlate and detect that a person tends to go to sleep later in the evening, wake later and take longer to ‘get going’ in the morning when they are more stressed. The ‘physiological sleep analysis’ might suggest ‘unstressed’ but a ‘behavioural sleep analysis’ might detect ‘stress behaviour’.

### The Cognitive Function Information

Where cognitive function information is used by the stress profiler 10, the accuracy and sensitivity of the stress profiler 10 generally increases when the cognitive function information includes measurements of more than one cognitive function parameter. Examples of different cognitive function measurements that may be used to provide cognitive function information include the results of memory tests, reaction-time measurements, measures of attention peripheral vision and comprehension, and the results of decision-making tests.

The cognitive function or performance tests can be in the form of online tasks, or interaction with smart watches, smart phones or other computing devices.

In one embodiment, the stress profiler 10 uses behavioural information obtained from measurements of memory tests, reaction-time tests and decision-making tests.

### Components of the Stress Profiler

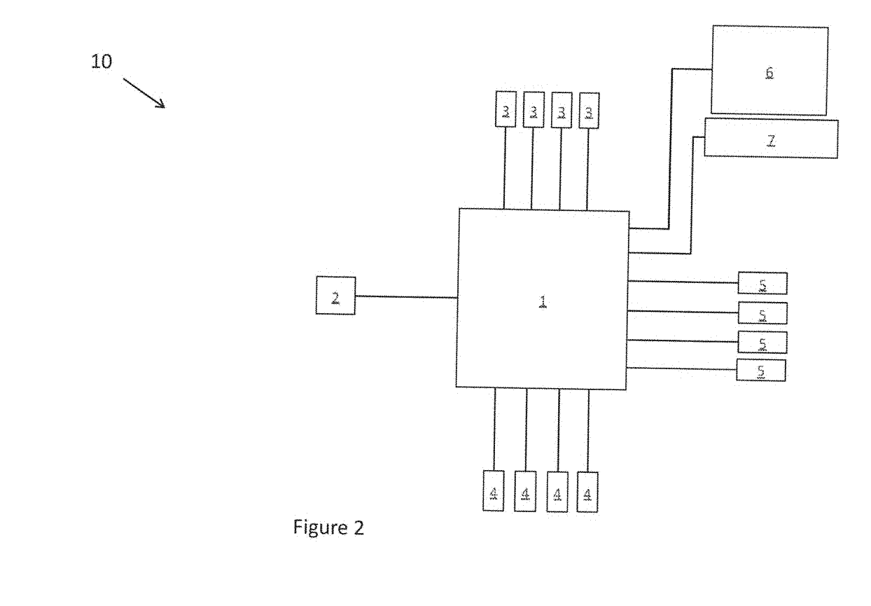

FIG. 2 is a block diagram of another representation of the components of the stress profiler 10 implemented in a computing device such as a smart phone, smart watch, tablet computer, desktop computer or laptop computer. The components are as follows:

1. processor

2. psychometric information collection tool

3. physiological information collection tool

4. behavioural information collection tool

5. cognitive function information collection tool

6. computer display for users (e.g. computer monitor, smart-phone LCD screen)

7. information entry interface for users (e.g. keyboard, mouse, touchscreen display surface).

These components are described in detail below.

### (1) Processor

The stress profiler 10 includes a processor (1) which receives stress information from the four types of information collection tools: psychometric information from the psychometric information collection tool (2); physiological information from the physiological information collection tool (3); behavioural information from the behavioural information collection tool (4), and cognitive function information from the cognitive function information collection tool (5). The processor processes this information to generate indicators of the magnitude and form of stress being experienced by the user. The processor includes software and CPU or GPU of the computing device.

Optionally, the processor includes functionality to read out information in a spoken voice to the user. Also, optionally, the processor includes a speech recognition function capable of recognizing the user's voice and verbal responses to questions.

### (2) Psychometric Information Collection Tool

The psychometric information collection tool presents questions the user on the display (6) and record the user's responses to those questions using the information entry interface (7). The questions are in relation to the user's subjective experience of stress.

Each question is displayed to the user one at a time. Multiple-choice answers are provided for users to choose from. There is no limit on the number of questions that can be asked, but the number of questions needs to be balanced against the total time it takes a user to respond to them. Depending the nature of the questions, around 30-40 questions may be sufficient. The questions should generally be quick to answer so that the process can completed in around 1-5 minutes.

As described above, to best obtain a psychometric stress measure a ‘long-form’ and ‘short form’ questionnaire has been developed as part of this invention. In use, the psychometric stress measure will be deployed in a two stage approach. During the first stage, an initial set of questions are posed to the individual (which will take approximately three minutes for the individual to answer). If the individual scores above a certain cut-off level, or in pre-set patterns, then the individual will be prompted to complete another block of questions, which constitutes the second stage of the questionnaire. The second set of questions will take the individual approximately four to five minutes to complete.

### (3) Physiological Information Collection Tool

The stress profiler 10 includes the ability to accept input from multiple physiological information collection tools (3). Each physiological information collection tool measures an aspect of the user's physiology which is indicative of stress in the user. Examples of suitable physiological information collection tools which can be used in the stress profiler 10 include, but are not limited to:
heart rate monitor, such as chest-mounted or arm-mounted devices used in sports e.g. Catapult Sports™ performance monitoring device, Polar™ heart rate monitor, Fitbit™, or smart watch capable of detecting heart rate;
respiratory rate monitor, such as chest-mounted or arm-mounted devices used in sports e.g. Catapult Sports™ performance monitoring device;
blood pressure monitor, such as a cuff around the upper arm which inflates and deflates periodically;
physical movement sensor, such as a gyroscope-enabled movement sensor used by sports people e.g. by Catapult Sports™;
location tracking device, such as a GPS-enabled smart phone or smart watch;
salivary cortisol analysis device;
skin conductivity measurement device;
skin temperature measurement device;
blood oxygen saturation measurement device e.g. finger-based pulse oximeter;
surface electromyography (surface EMG) device;
electroencephalography (EEG) device;
‘smart clothing’, including pants/trousers, underwear, socks, shoes, shirts/T-shirts, gloves, hats/caps/helmets, glasses, watches, smart-watches, wrist and ankle bands, as well as adhesive patches, embedded with various sensors, including electrical signal, conductivity (galvanic conductance and resistance), accelerometers, force, temperature, chemical sensors and nanotechnology sensors can be used to provide physiological information;
Nanotechnology sensors, which can include single-blood drop devices, transdermal patches, subcutaneous or circulatory injectable devices;
blood testing apparatus (e.g. suitable for detecting chemicals, molecules, proteins and hormones indicative of stress or stimulation of the hypothalamo-pituitary-adrenal axis (the HPA Axis) such as catecholamines, epinephrine (adrenalin), norepinephrine (noradrenaline), serotonin, or dopamine); and
human-implanted chip or wires (e.g. suitable for detecting chemicals, molecules, proteins and hormones indicative of stress or stimulation of the hypothalamo-pituitary-adrenal axis (the HPA Axis) such as catecholamines, epinephrine (adrenalin), norepinephrine (noradrenaline), serotonin, or dopamine).

The tools (3) may be either integrated into the computing device, online or a standalone external device. Where a tool is external, it can be connected to the computing device by any suitable method, such as by cable or a wireless Bluetooth connection.

### (4) Behavioural Information Collection Tool

The stress profiler 10 includes the ability to accept input from multiple behavioural information collection tools. Each behavioural information collection tool measures an aspect of the user's behaviour which is indicative of stress in the user. Examples of suitable behavioural information collection tools which can be used in the stress profiler 10 include, but are not limited to:
eye-tracking software;
a location tracking device, such as a GPS-enabled smart phone or smart watch;
Bluetooth tracking software to track the nearby presence of devices owned by other individuals;
internet browsing history analysis software;
smartphone, smart-watch or other wearable device, tablet or computer accelerometers, gyroscopes or altimeters,
proximity sensing devices such as NFC, Wi-Fi or Bluetooth, particularly with enhanced location specificity, (proximity to other devices, enhanced location specificity),
keystroke rate, cadence, typing style, pressure or ‘force’ detection (keypad, trackpad, screen pressure sensor);
voice analysis (tone, cadence, word and phrase detection), phone usage, including call time, numbers dialed, time of day calls placed,
application (‘app’) usage, including specific applications used, duration of usage, time of day apps used, in-app analytics (use characteristics within any app), keyword searches, word and phrase usage (usually applied within word processing, email, messaging and social media applications but not limited to these), gait and posture analysis and purchasing history;
car/driving/riding style, including steering inputs, acceleration, deceleration, braking, speed of driving, brake and accelerator force, door pressure sensors and other vehicle sensors;
home or office sensors, which can measure movement, body temperature, television usage (channels watched, time watching, eye movement), refrigerator analytics, heating and cooling analytics and other ‘smart home’ analytics;
bicycle meters (pedal force, pedaling cadence, acceleration, speed, routes taken, GPS, altimeter, time on bicycle, and so on), pedometers, gait analysis measures; and
‘smart clothing’, which includes pants/trousers, underwear, socks, shoes, shirts/T-shirts, gloves, hats/caps/helmets, glasses, watches, smart-watches, wrist and ankle bands, as well as adhesive patches

The stress profiler 10 first requests permission from the user to collect behavioural information, and then routinely collects the information in the background without interrupting the user.

The tools (4) may be either integrated into the computing device, online or a standalone external device. Where a tool is external, it can be connected to the computing device by any suitable method, such as by cable or a wireless Bluetooth connection.

### (5) Cognitive Function Information Collection Tool

The stress profiler 10 includes the ability to accept input from multiple cognitive function information collection tools. Each cognitive function information collection tool measures an aspect of the user's cognitive function which is indicative of stress in the user. Examples of suitable cognitive function information collection tools which can be used in the stress profiler 10 include, but are not limited to:
software to test the memory of a user;
software to test the reaction time of a user;
software to test the attention, peripheral vision and comprehension of a user;
software to test the decision-making ability of a user.

The processor (1) prompts the user to complete one or more of the cognitive function tests. If the user agrees to do the test(s), the processor presents the user with a brief cognitive function test. The test should generally be quick to do, and perhaps take from 5 seconds to 2 minutes to complete. The memory test may prompt the user at a later time to remember a piece of information.

The tools (5) may be either integrated into the computing device, online or a standalone external device. Where a tool is external, it can be connected to the computing device by any suitable method, such as by cable or a wireless Bluetooth connection.

### The Algorithm in the Processor

The processor (1) uses an algorithm to generate a personal stress profile which is indicative of the magnitude and form of stress experienced by the user at the time of testing. Stress can be measured and categorized in various ways. When applied consistently, the algorithm highlights relative differences over time for each individual, and differences from one individual to another. The stress profile can also be used as a basis to test the effectiveness of different types of stress treatment on each form of stress.

The profile in this embodiment identifies and quantifies four major domains or forms of stress:
1. physical/physiological stress,
2. mental stress,
3. emotional stress, and
4. current perceived life stress.

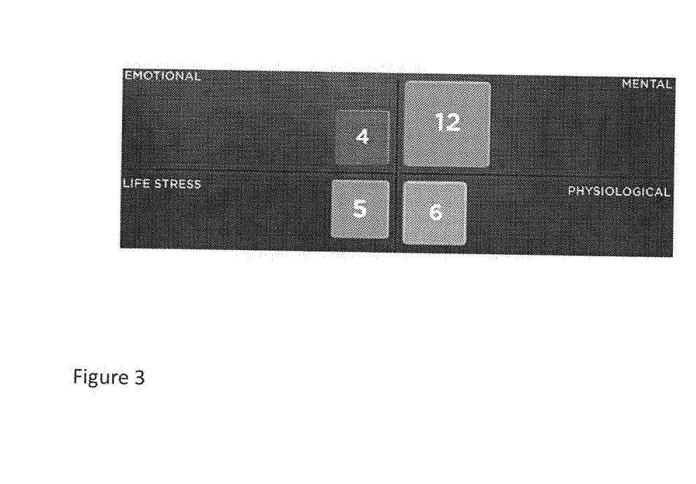

At the conclusion of stress profile measurement, the processor generates a graphical representation of the results in the form of a chart with four quadrants (see FIG. 3). Each quadrant of the chart shows a stress score for a different the form of stress. Each score is indicative of the magnitude of that form of stress. At a glance, one can graphically see the magnitude and form of stress being experienced by a user. In the example shown in FIG. 3, all four forms of stress are present but the mental stress is greatest. The mental stress score is 12, whereas the physiological stress score is 6, the life stresses score is 5 and the emotional stress score is 4.

The magnitude of each form of stress is calculated by determining a score for each form of stress. The inputs for each score are described qualitatively below.

### 1. Physical/Physiological Stress Score

The physical/physiological stress score is calculated from two scores:

### a) Physical Stress Score

The stress information used to calculate this score comprises:
Psychometric information indicative of physical stress
Physiological information indicative of physical stress
Behavioural information indicative of physical stress

### b) Physiological Stress Score

The stress information used to calculate this score comprises:
Psychometric information indicative of physiological stress
Physiological information indicative of physiological stress
Behavioural information indicative of physiological stress

### 2. Mental Stress Score

The mental stress score is calculated from two scores:

### a) Memory Stress Score

The stress information used to calculate this score comprises:
Psychometric information indicative of memory stress
Physiological information indicative of memory stress
Behavioural information indicative of memory stress
Cognitive function information indicative of memory stress

### b) Cognitive Function Stress Score

The stress information used to calculate this score comprises:
Psychometric information indicative of cognitive function stress
Physiological information indicative of cognitive function stress
Behavioural information indicative of cognitive function stress
Cognitive function information indicative of cognitive function stress

### 3. Emotional Stress Score

The emotional stress score is calculated from four scores:

### a) Irritability or Reactivity Stress Score

The stress information used to calculate this score comprises:
Psychometric information indicative of irritability or reactivity
Physiological information indicative of irritability or reactivity
Behavioural information indicative of irritability or reactivity

### b) Ability to Rest/Sleep Stress Score

The stress information used to calculate this score comprises:
Psychometric information indicative of ability to rest/sleep
Physiological information indicative of ability to rest/sleep
Behavioural information indicative of ability to rest/sleep

### c) Perceived Personal Efficacy Stress Score

The stress information used to calculate this score comprises:
Psychometric information indicative of perceived personal efficacy
Physiological information indicative of perceived personal efficacy
Behavioural information indicative of perceived personal efficacy

### d) Perceived Workplace Efficacy Stress Score

The stress information used to calculate this score comprises:
Psychometric information indicative of perceived workplace efficacy
Physiological information indicative of perceived workplace efficacy
Behavioural information indicative of perceived workplace efficacy

### 4. Current Perceived Life Stress

The current perceived life stress score is calculated from two scores:

### a) Perceived Stress in Life Stress Score

The stress information used to calculate this score comprises:
Psychometric information indicative of perceived stress in life
Physiological information indicative of perceived stress in life
Behavioural information indicative of perceived stress in life

### b) Perceived Work Stress Score

The stress information used to calculate this score comprises:
Psychometric information indicative of work stress
Physiological information indicative of work stress
Behavioural information indicative of work stress

### Acute Stress Score

The processor also generates an acute stress score which is indicative of the magnitude of acute stress. This score is calculated from aspects of the stress information (psychometric information, physiological information, behavioural information and cognitive function information) which are indicative of acute stress.

### Chronic Stress Score

The processor also generates a chronic stress score which is indicative of the magnitude of chronic stress. This score is calculated from aspects of the stress information (psychometric information, physiological information, behavioural information and cognitive function information) which are indicative of chronic stress.

### Resilience to Stress Indicator

The processor can also measure a person's response to an acute stress event or an acute stress state, and generate a measure of stress resilience. This can be a score, which is indicative of the time taken for the individual acute stress elements and indicators, either singular or in combination, to rise in response to an acute stress (speed of response to acute stress). It can also be a score, which is indicative of the level to which the individual acute stress elements and indicators, either singular or in combination, reach after an acute stress (intensity of response to acute stress). It can also be a score, which is indicative of the time taken for the individual acute stress elements and indicators, either singular or in combination, to return to ‘unstressed’ or baseline levels following any particular stressful event (speed of resolution).

Further, when an individual is becoming overwhelmed or ‘burnt out’ by a singular stress or chronic stress, their acute stress response can become diminished. They can have a delayed or reduced response of some acute stress response components to an acute stress such as exercise (sluggish or minimal response) that should ordinarily elicit a stress response.

### Psychological Condition Indicator

Behaviour is a very accurate predictor/identifier of an individual's internal or psychological state. The data obtained from the physiological and behavioural analytic elements of the system and method of the present invention is capable of detecting several common and debilitating psychological conditions.

For example, the following psychological conditions are exemplary (but not exhaustive) of the types of disorders and conditions that can be predicted or identified by the system and method of the present invention, post-traumatic stress disorder, depression, anxiety, suicide/self-harm risk or prediction, bipolar disorder, attention deficit hyperactivity disorder, sleep disorders, addictive traits and physical abuse or the likelihood of such (be it victim or perpetrator).

If the physiological and behavioural elements detect the likelihood of a specific psychological condition, the system and method of the present invention would automatically prompt the individual to complete a relevant psychometric measure (or measures) related to the identified psychological condition.

As an example, if the behavioural analytic measures suggested a likelihood of depression, one or more specific depression psychometric questionnaires would be prompted and the user would be asked to complete them.

### EXAMPLES

If a person scores more highly on the memory stress score (which contributes to the mental stress score), the person may be best helped by interventions that are more suited to assisting memory, for example psychotherapy, meditation, mindfulness, memory training applications, training in time and memory management systems, and so on. Conversely massage may not be the first choice of intervention.

Alternatively, if a person shows a high physical stress score (which contributes to the physical/physiological stress score), then an appropriate intervention may be yoga, massage, exercise.

### Embodiment 1

This embodiment is a mobile version of the stress profiler 10 designed to operate on a smart phone, smart watch or tablet computing device. The processor includes a mobile app. Some of the stress information is collected by the app in the background without any manual input by the user, and the remainder of the information requires active participation of the user. However, not all information is collected automatically. To generate a stress profile the user needs to open for the stress profiler 10 the mobile app and perform a series of tests managed by the app. The app manages a range of stress information collection tools, which are implemented as follows.

### Psychometric Information Collection Tool

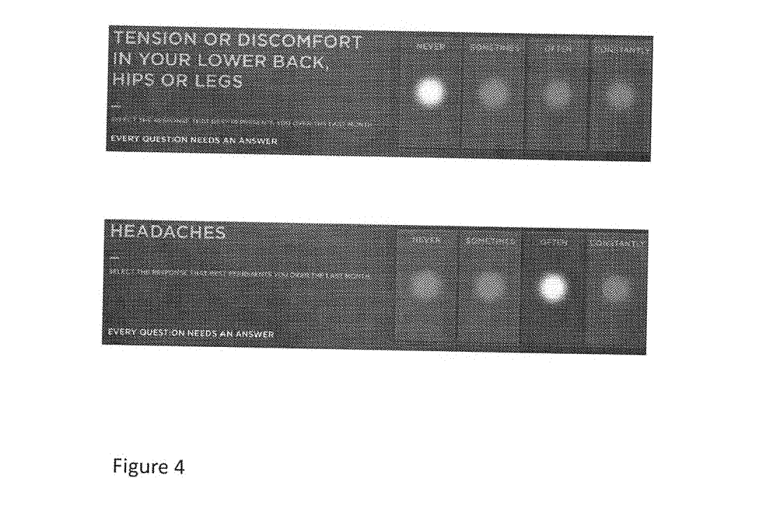

The psychometric information collection tool prompts the user to answer a series of questions. Every question must be answered. Each question has multiple-choice answers. A separate button is provided for each answer, as shown in FIG. 4.

### Example of Questions:

1. Do you have tension or discomfort in your lower back, hips or legs?
Multiple-choice answers: Never, Sometimes, Often, Constantly
2. Do you have headaches?
Multiple-choice answers: Never, Sometimes, Often, Constantly

### Physiological Information Collection Tools

The physiological information collection tool comprises software which controls the device's camera to image the user's face and thereby detect:
pulse;
skin colour and circulation;
facial expression.

When an external heart rate monitor is connected (e.g. Catapult Sports' performance monitoring device) the physiological information collection tool uses it to measure:
heart rate;
heart rate variability;
respiratory rate;
respiratory depth.

### Cognitive Function Information Collection Tools

The cognitive function information collection tools comprise cognitive function tests, such as memory tests, reaction time tests, and decision-making tests. The stress profile app selects the tests appropriate to the user and guides the user through the tests.

### Behavioural Information Collection Tools

The stress profiler 10 includes a number of behavioural information collection tools:
software which uses the GPS and accelerometer to detect movement and location, sleep cycle detection (if phone is placed in the bed);
software which detects nearby Bluetooth, NFC, Wi-Fi, and thereby detects places and people nearby;
software which uses the camera to detect the direction and speed of eye movements, and determines the time spent on certain ‘news articles’ and reading tasks;
software which analyses Internet search history, app usage, key word dominance when within particular apps or websites such as social media;
software which analyses purchasing characteristics based on smartphone-enabled purchasing or Internet purchase history information.
This embodiment of the stress profiler 10 gives immediate feedback to users about their stress profile and alerts the user appropriately. The stress profiler 10 also suggests actions the user may take to manage their measured stresses.

### Embodiment 2

This embodiment includes all of the features of embodiment 1, plus additional stress information collection tools.

### Additional Physiological Information Collection Tools

The stress profiler 10 is capable of receiving and processing information from a more sophisticated external device (e.g. Catapult Sports monitor) which directly measures:
movement and physical balance;
respiratory rate and depth;
ECG;
skin temperature and conductivity;
sleep cycle.

The stress profiler 10 is also capable of receiving and processing information from an external blood oxygen saturation measurement device such as a finger-top oximeter.

### Additional Behavioural Information Collection Tools

The stress profiler 10 is capable of receiving and processing information from the user's car, home or computer. The home and car information may be indicative of the user's movement, acceleration, deceleration, time spent doing certain tasks, types of tasks completed at home, types of foods consumed (e.g. via an intelligent fridge). The user's computer information will be of the same type of behavioural information collected on the mobile device e.g. Internet search history, app usage, key word dominance when within particular apps or websites such as social media.

### Embodiment 3

This embodiment includes all of the features of embodiment 2, plus additional stress information collection tools.

### Additional Physiological Information Collection Tools

The stress profiler 10 is capable of receiving and processing additional information from other sources, including:
blood analysis information (from a lab, mobile testing kit or integrated test) for markers of stress.
urine analysis information (from a lab, mobile testing kit or integrated test) for markers of stress.
saliva analysis information (from a lab, mobile testing kit or integrated test) for markers of stress.
EEG measurements from self-administered EEG (e.g. skin-applied patches and wires, or with a cap).
skin conductance and skin trace chemical detection (from a lab, mobile testing kit or integrated test or skin-applied patch) for evidence of sweat levels or chemicals found on skin (secreted through skin or through perspiration).
blood pressure from an external blood pressure monitor.
DNA and hair analysis information (from a lab, mobile testing kit or integrated test) for disposition towards stress response and stress history markers.

The specific relationships between the stress information and the stress scores are all determined empirically by correlating the results of psychometric testing with the results of physiological testing, cognitive function testing and behavioural testing. In order to identify widespread correlations, a large set of information must be generated by testing a large number of people, for example 10,000 people or more.

For each stress score, the contributing measurements are each given a weighting depending on the relative impact on the stress score.

### Example: Physical Stress Score

An example of a formula for calculating the physical stress score is below.

The variables a, b, c . . . below are weighting co-efficients determined from widespread correlations identified by testing a large number of people e.g. 10,000 people.

The ‘ratings’ are determined for each individual by first measuring baselines to establish what is normal for them. For example, it is normal for salivary cortisol levels to vary over the course of a day, and from person to person, so it is necessary to measure a baseline at a particular time of day to determine what is normal for an individual.
Physical stress score=
   psychometric physical test score+
   a (heart rate−80)+
   b (heart rate variability rating)+
   c (respiratory rate−16)+
   d (respiratory rate variability rating)+
   e (skin conductivity rating)+
   f (temperature−37.2)+
   g (skin temperature−upper range of normal temperature for that site)+
   h (systolic blood pressure measure−120 mmHg)+
   i (diastolic blood pressure measure−80 mmHg)+
   j (EMG rating)+
   k (EEG rating)+
   l (sleep rating)+
   m (salivary cortisol rating)+
   n (ACTH measure−ACTH normal)+
   o (movement balance rating)+
   p (gyroscope measurement rating)+
   q (acceleration measurement rating)

Now that embodiments have been described, it will be appreciated that some embodiments have some of the following advantages:
The outcome of a stress measurement may not be binary, i.e. either stressed or not stressed, and may provide specific tailored advice that may lead to better and faster outcomes.
Embodiments may define whether a subject is more emotionally stressed or mentally stressed and as such may be able to shed light on a person's state and may be able to be prescriptive and inform that person about which interventions are more likely to be of assistance.
Hit and miss outcomes may be reduced. An individual that is chronically very mentally stressed may be given a targeted approach, rather than a shot gun approach, not simply being provided with a list of possible stress relieving actions from which to select and try. For example, out of possible options including yoga, exercise, squeezing a stress ball, mindfulness, meditation, psychotherapy, and so on embodiments may select an action most likely to be beneficial.
Relatively high precision in recommendations may reduce delayed effective intervention, wasted efforts and expense and result in better outcomes.

Variations and/or modifications may be made to the embodiments described without departing from the spirit or ambit of the invention. The present embodiments are, therefore, to be considered in all respects as illustrative and not restrictive.

Prior art, if any, described herein is not to be taken as an admission that the prior art forms part of the common general knowledge in any jurisdiction.

In the claims which follow and in the preceding description of the invention, except where the context requires otherwise due to express language or necessary implication, the word “comprise” or variations such as “comprises” or “comprising” is used in an inclusive sense, that is to specify the presence of the stated features but not to preclude the presence or addition of further features in various embodiments of the invention.

1. A method for generating stress level information for an individual, the method comprising the step of:
in a processor, processing stress information for the individual comprising at least two of psychometric information for the individual, physiological information for the individual, behavioural information for the individual, and cognitive function information for the individual.

2. A method defined by claim 1 comprising the step of receiving the stress information.

3. A method defined by claim 1 wherein the step of processing the stress information comprises the step of correlating at least one stress indicator in one of the psychometric information, the physiological information, the behavioural information, and the cognitive function information with at least one other stress indicator in at least one other of the psychometric information, the physiological information, the behavioural information, and the cognitive function information.

4. A method defined by claim 1 wherein the step of processing the stress information comprises the step of determining a stress feature that recurs within the stress information.

5. A method defined by claim 4 comprising the steps of receiving further stress information for the individual, and confirming the presence of the stress feature within the further stress information.

6. A method defined by claim 5 comprising the step of generating an alert indicative of the presence of the stress feature when the presence of the stress feature within the further stress information is so confirmed.

7. A method defined by claim 1 wherein the stress information comprises the psychometric information.

8. A method defined by claim 7 comprising the step of generating the psychometric information by the individual responding to an electronic stress questionnaire.

9. A method defined by claim 8 wherein the questionnaire is in two parts, each comprising a different set of predefined questions, whereby the individual is presented with the second set of questions based on predetermined criteria correlating with the answers provided to the first set of questions.

10. A method defined by claim 1 wherein the psychometric information is indicative of a plurality of chronic stress indicators.

11. A method defined by claim 1 wherein the stress information comprises the physiological information.

12. A method defined by claim 11 comprising the step of generating the physiological information.

13. A method defined by claim 12 wherein the step of generating the physiological information comprises the step of generating information for each of a plurality of physiological functions in the individual.

14. A method defined by claim 13 wherein the step of generating information indicative of stress in each of a plurality of physiological functions in the individual comprises generating at least one of heart rate information, heart rate variability information, respiratory rate information, respiratory rate variability information, blood pressure information, physical movement information, cortisol level information, a skin conductivity information, skin temperature information, skin or hair analysis, DNA analysis, blood oxygen saturation information, surface electromyography information, electroencephalography information, blood information, saliva information, and urine information.

15. A method defined by claim 1 wherein the stress information comprises the behavioural information.

16. A method defined by claim 4 comprising the step of generating the behavioural information.

17. A method defined by claim 16 wherein the step of generating the behavioural information comprises at least one of the steps of:
generating eye movement information indicative of eye movement of the individual;
generating location information indicative of a plurality of locations the individual has been;
generating nearby device information indicative of the nearby presence a plurality of devices of a plurality of people to the individual;
generating internet browsing history information for the individual;
generating keystroke rate, cadence, typing style, pressure or ‘force’ detection information for the individual;
generating voice analysis, including tone, cadence, word and phrase detection information for the individual;
generating telephone usage analysis, including call time, numbers dialed and time of day calls placed information for the individual;
generating driving style, including steering inputs, acceleration, deceleration, braking, speed of driving, brake and accelerator force and data from door pressure sensor information for the individual;
generating movement, body temperature, television usage, including channels watched, time watched and eye movement whilst watching, refrigerator analytics, heating and cooling analytics information for the individual;
generating bicycle data, including pedal force, pedaling cadence, acceleration, speed, routes taken, GPS data, altimeter data, time on bicycle, pedometer data information for the individual;
generating pedometer data and gait analysis information for the individual;
generating application usage information indicative of application usage by the individual;
generating media consumption information indicative of media consumption by the individual;
generating spending behaviour information indicative of the individual's spending behaviour;
generating food choice information indicative of a plurality of food choices made by the individual;
generating social outing information indicative of the individual's social outing activity;
generating productivity information indicative of the individual's ability to work and be productive; and
generating leave information indicative of leave taken by the individual.

18. A method defined by claim 1 wherein the stress information comprises the cognitive function information.

19. A method defined by claim 18 comprising the step of generating the cognitive function information.

20. A method defined by claim 19 wherein the step of generating the cognitive information comprises at least one of the steps of:
generating memory function information indicative of a memory function of the individual;
generating reaction time information indicative of a reaction time of the individual;
generating attention ability, peripheral vision and comprehension ability of the individual; and
generating decision-making ability information indicative of a decision-making ability of the individual.

21. A method defined by claim 1 wherein the step of processing the stress information comprises the steps of generating:
a physiological and/or physical stress score for the individual using the stress information;
a mental stress score for the individual using the stress information;
an emotional stress score for the individual using the stress information;
a life stress score for the individual using the stress information.

22. A method defined by claim 21 comprising the step of displaying on an electronic display graphical representations of:
the physiological and/or physical stress score for the individual;
the mental stress score for the individual;
the emotional stress score for the individual; and
the life stress score for the individual.

23. A method defined by claim 21 wherein the step of displaying on an electronic display comprises the step of displaying a token for each of:
the physiological and/or physical stress score;
the mental stress score;
the emotional stress score; and
the life stress score;
wherein the size of each token is indicative of a respective stress score magnitude.

24. A method defined by claim 23 wherein the token for each of the physiological and/or physical stress score, the mental stress score, the emotional stress score; and the life stress score are simultaneously displayed

25. A method defined by claim 1 comprising the step of generating an acute stress score indicative of a magnitude of acute stress for the individual.

26. A method defined by claim 25 comprising the step of generating a stress resilience score indicative of a response to acute stress for the individual.

27. A method defined by claim 26 wherein the stress resilience score is indicative of one or more of the time taken for the individual to respond to an acute stress event, if the individual exhibits any response to an acute stress event, and if so, the level of response exhibited by the individual to an acute stress event and the time taken for the individual's stress information to return to baseline levels following a period of acute stress.

28. A method defined by claim 1 comprising the step of generating a chronic stress score indicative of a magnitude of chronic stress for the individual.

29. A method defined by claim 1 wherein the stress level information can indicate the individual is suffering from a psychological condition, including post-traumatic stress disorder, depression, anxiety, suicide/self-harm risk or prediction, bipolar disorder, attention deficit hyperactivity disorder, sleep disorders and addictive traits.

30. A system for generating stress level information for an individual, the system comprising:
a stress information processing module configured to process stress information for the individual, the stress information for the individual comprising at least two of psychometric information for the individual, physiological information for the individual, behavioural information for the individual, and cognitive function information for the individual.

31. A system defined by claim 30 comprising a stress information receiver configured to receive the stress information.

32. A system defined by claim 30 wherein the stress information processing module is configured to correlate at least one stress indicator in one of the psychometric information, the physiological information, the behavioural information, and the cognitive function information with at least one other stress indicator in at least one other of the psychometric information, the physiological information, the behavioural information, and the cognitive function information.

33. A system defined by claim 30 wherein the stress information processing module is configured to determine a stress feature that recurs within the stress information.

34. A system defined by claim 33 wherein the stress information processing module is configured to confirm the presence of the stress feature within further stress information for the individual.

35. A system defined by claim 34 comprising an alert generation module configured to generate an alert indicative of the presence of the stress feature when the presence of the stress feature within the further stress information is so confirmed.

36. A system defined by claim 30 wherein the stress information comprises the psychometric information.

37. A system defined by claim 30 comprising a psychometric information generator configured to generate psychometric information for the individual.

38. A system defined by claim 37 wherein the psychometric information generator is configured to execute an electronic psychometric questionnaire for the individual.

39. A system defined by claim 38 wherein the questionnaire is in two parts, each comprising a different set of predefined questions, whereby the individual is presented with the second set of questions based on predetermined criteria correlating with the answers provided to the first set of questions.

40. A system defined by claim 30 wherein the psychometric information is indicative of a plurality of chronic stress indicators.

41. A system defined by claim 30 wherein the stress information comprises the physiological information.

42. A system defined by claim 30 comprising a physiological information generation system configured to generate the physiological information.

43. A system defined by claim 42 wherein the physiological information generation system is configured to generate information for each of a plurality of separate physiological functions in the individual.

44. A system defined by claim 43 wherein the physiological information generation system is configured to generate information for each of a plurality of separate physiological functions in the individual is configured to generate at least one of a heart rate information, heart rate variability information, respiratory rate information, respiratory rate variability information, blood pressure information, physical movement information, cortisol level information, skin conductivity information, skin temperature information, skin or hair analysis, DNA analysis, blood oxygen saturation information, surface electromyography information, electroencephalography information, blood measurement information, saliva measurement information, and urine measurement information.

45. A system defined by claim 30 wherein the stress information comprises the behavioural information.

46. A system defined by claim 30 comprising a behavioural information generator configured to generate the behavioural information.

47. A system defined by claim 46 wherein the behavioural information generator comprises at least one of:
an eye movement information generator configured to generate eye movement information indicative of eye movement of the individual;
a location information generator configured to generate location information indicative of a plurality of locations the individual has been;
a nearby device information generator configured to generate nearby device information indicative of the nearby presence a plurality of devices of a plurality of people to the individual
an internet browsing history generator configured to generate internet browsing history for the individual;
a keystroke generator configured to generate rate, cadence, typing style, pressure or ‘force’ detection information for the individual;
a voice analysis generator configured to generate tone, cadence, word and phrase detection information for the individual;
a telephone usage analysis generator configured to generate call time, numbers dialed and time of day calls placed information for the individual;
a driving style generator configured to generate steering inputs, acceleration, deceleration, braking, speed of driving, brake and accelerator force and data from door pressure sensor information for the individual;
a movement generator configured to generate body temperature, television usage, including channels watched, time watched and eye movement whilst watching, refrigerator analytics, heating and cooling analytics information for the individual;
a bicycle usage data generator configured to generate pedal force, pedaling cadence, acceleration, speed, routes taken, GPS data, altimeter data, time on bicycle, pedometer data information for the individual;
a pedometer and gait analysis generator configured to generate pedometer data information for the individual;
an application usage information generator configured to generate information indicative of application usage by the individual;
a media consumption information generator configured to generate media consumption information indicative of media consumption by the individual;
a spending behaviour information generator configured to generate spending behaviour information indicative of the individual's spending behaviour;
a food choice information generator configured to generate food choice information indicative of a plurality of food choices made by the individual;
a social outing information generator configured to generate social outing information indicative of the individual's social outing activity;
a productivity information generator indicative of the individual's ability to work and be productive; and
a leave information generator configured to generate leave information indicative of leave taken by the individual.

48. A system defined by claim 30 wherein the stress information comprises the cognitive function information.

49. A system defined by claim 30 comprising a cognitive function generator configured for generating the cognitive function information.

50. A system defined by claim 49 wherein the cognitive function generator comprises at least one of:
a memory function information generator configured to generate memory function information indicative of a memory function of the individual;
a reaction time information generator configured to generate reaction time information indicative of a reaction time of the individual;
an attention ability, peripheral vision capability and comprehension ability generator to generate information indicative of the ability of the individual; and
a decision-making ability information generator configured to generate decision-making ability information indicative of a decision-making ability of the individual.

51. A system defined by claim 30 wherein the stress information processing module is configured to generate at least one of:
a physiological and/or physical stress score for the individual using the stress information;
a mental stress score for the individual using the stress information;
an emotional stress score for the individual using the stress information;
a life stress score for the individual using the stress information.

52. A system defined by claim 51 comprising a display and the configured to display on the electronic display graphical representations of:
the physiological and/or physical stress score for the individual;
the mental stress score for the individual;
the emotional stress score for the individual; and
the life stress score for the individual.

53. A system defined by claim 30 configured to display on the electronic display a token for each of:
the physiological and/or physical stress score;
the mental stress score;
the emotional stress score; and
the life stress score;
wherein the size of each token is indicative of a respective stress score magnitude.

54. A system defined by claim 30 configured to simultaneously display the token for each of the physiological and/or physical stress score, the mental stress score, the emotional stress score, and the life stress score.

55. A system defined by claim 30 wherein the stress information processing module is configured to generate an acute stress score indicative of a magnitude of acute stress for the individual.

56. A system defined by claim 55 wherein the stress information includes a stress resilience score indicative of a response to acute stress for the individual.

57. A system defined by claim 56 wherein the stress resilience score is indicative of one or more of the time taken for the individual to respond to an acute stress event, if the individual exhibits any response to an acute stress event, and if so, the level of response exhibited by the individual to an acute stress event and the time taken for the individual's stress information to return to baseline levels following a period of acute stress.

58. A system defined by claim 30 wherein the stress information processing module is configured to generate a chronic stress score indicative of a magnitude of chronic stress for the individual.

59. A system defined by claim 30 wherein the stress level information can indicate the individual is suffering from a psychological condition, including post-traumatic stress disorder, depression, anxiety, suicide/self-harm risk or prediction, bipolar disorder, attention deficit hyperactivity disorder, sleep disorders and addictive traits.

60. Non-transitory processor readable tangible media including program instructions which when executed by a processor causes the processor to perform a method defined by any one of the claims 1 to 29.

61. Computer program for instructing a processor, which when executed by the processor causes the processor to perform a method defined by any one of the claims 1 to 29.
